(19) United States
(12) Patent Application Publication
(10) Pub. No.: US 2026/0325172 A1
(43) Pub. Date: Oct. 22, 2026

(54) DEVICE FOR PERIURETHRAL BULKING AND METHODS INCORPORATING THE SAME

(71) Applicant: Thomas Jefferson University, Philadelphia, PA (US)

(72) Inventor: Karolynn Teresa Echols, Philadelphia, PA (US)

(21) Appl. No.: 19/105,143

(22) PCT Filed: Aug. 21, 2023
(86) PCT No.: PCT/US2023/030694

§ 371 Date: Feb. 20, 2025

Related U.S. Application Data
(60) Provisional application No. 63/399,897, filed on Aug. 22, 2022.

(51) Int. Cl. A61B 17/34; A61B 1/018; A61B 1/307; A61M 25/00

(52) U.S. Cl. CPC A61B 17/3478; A61B 1/018; A61B 1/307; A61B 17/3468; A61M 25/0084; A61M 2025/0086

(57) ABSTRACT

A device for periurethral bulking is provided. The device includes a distal end comprising a sheath member having a optically transparent tip. The sheath member is configured to include: a distal portion of a cystoscope; and a plurality of needles, wherein each of the plurality of needles includes a respective pathway. The device also includes a proximal end comprising viewing optics of the cystoscope. The device also includes a bridge attachment positioned in between the distal end and the proximal end, wherein the bridge attachment is in fluidic communication with the plurality of needles. The optically transparent tip comprises a plurality of outlet channels which allow passage of only a single needle. Each of the plurality of outlet channels is configured to guide a respective needle to an angle of about 45-90 degrees. Each needle and the respective pathway of the plurality of needles are bendable to about 45-90 degrees.

### CROSS-REFERENCE TO RELATED APPLICATIONS

This application claims priority under 35 U.S.C. § 119(e) to U.S. Provisional Patent Application No. 63/399,897, filed Aug. 22, 2022, the disclosures of which is incorporated herein by reference in its entirety.

### FIELD OF INVENTION

The present invention is directed to a device for use in medical procedures, including for periurethral bulking.

### BACKGROUND

In the medical field, stress urinary incontinence (SUI) is defined as leakage of urine during any activity that causes an increase in intraabdominal pressure. Patients complain of leakage of urine in connection with coughing, sneezing, laughing, running, other modalities of exercise, and sexual activities. Up to 35% of women have SUI which contributes to social health consequences (e.g., isolation, depression, etc.). Of the treatments for SUI, the most common and most successful procedure (approximately 80-90%) involves a permanent polypropylene mesh, which has a serious stigma to it (and is no longer approved by the FDA for transvaginal repair of pelvic organ prolapse (POP)). This stigma gives patients angst in choosing this type of treatment for their SUI.

Overactive bladder (OAB) or urinary urgency, frequency, nocturia, is defined as a sudden need to urinate with or without leakage. OAB can be more common in women than in men.

Depending on age and gender, the prevalence rates have been seen in almost half of populations studied. Although there are treatment options for this condition, the options can be time consuming, invasive and/or have undesirable side effects. Thus, there exists a need in the art to develop treatment options for OAB or urinary urgency. The present invention addresses this unmet need.

### SUMMARY

One aspect of the invention provides a device for periurethral bulking, including a distal end comprising a sheath member having a optically transparent tip. The sheath member is configured to include: a distal portion of a cystoscope; and a plurality of needles, wherein each of the plurality of needles includes a respective pathway. The device also includes a proximal end comprising viewing optics of the cystoscope. The device also includes a bridge attachment positioned in between the distal end and the proximal end, wherein the bridge attachment is in fluidic communication with the plurality of needles. The optically transparent tip comprises a plurality of outlet channels such that each of the plurality of outlet channels allows passage of only a single needle from the plurality of needles. Each of the plurality of outlet channels is configured to guide a respective needle to an angle of about 45-90 degrees relative to the sheath member. Each needle and the respective pathway of the plurality of needles are bendable to an angle such that each needle and the respective pathway are configured to deploy into a tissue of a subject at an angle of about 45-90 degrees relative to the sheath member.

Another aspect of the invention provides a method for viewing and simultaneously injecting a bulking material at multiple locations in the urethra of a subject. The method includes the steps of: (a) injecting a bulking agent into a urethra of the subject using the device described herein (i.e., any embodiment described herein).

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views. It should be understood, however, that the invention is not limited to the precise arrangements and instrumentalities of the embodiments shown in the drawings.

Throughout the various drawings provided herein, the same reference characters refer to the same element (and similar reference characters, such as those ending with an “a”, “b”, or “c”, refer to substantially similar elements). Thus, a description of certain elements may be omitted (or limited) in connection with some of the drawings.

FIGS. 1A-1H illustrate various views of a device for periurethral bulking, in accordance with embodiments of the disclosure. 1A-1C illustrate a side view of a device for periurethral bulking in an undeployed configuration (FIG. 1A), a side view of a distal end portion thereof (FIG. 1B), and a cross-sectional side view of the distal end (FIG. 1C). 1D-1H illustrate a side view of a device for periurethral bulking in a deployed configuration (FIG. 1D), a distal end portion thereof (FIG. 1E), a cross-sectional side view of the distal end (FIG. 1F), a perspective view of the device (FIG. 1G), and a perspective view of the distal end portion thereof (FIG. 1F). FIG. 2 illustrates an exploded perspective view of a device for periurethral bulking, in accordance with an exemplary embodiment of the disclosure.

FIGS. 3A-3D illustrate various cross-sectional views of a device in accordance with an exemplary embodiment of the disclosure. 3A shows a cross-sectional side view of a device. 3B and 3C each illustrate a detailed cross-sectional side view of a fluid injection system of a device. 3D illustrates a partial cross-sectional perspective view of a needle manifold and related elements, in accordance with an exemplary embodiment of the disclosure.

FIGS. 4A-4C illustrate various devices for periurethral bulking in accordance with various exemplary embodiments of the disclosure. 4A illustrates a side view of a full device. 4B and 4C each illustrate a cross-sectional side view of a distal end portion of a device for periurethral bulking in an undeployed configuration.

FIGS. 5A-5D illustrate various side views of a device for periurethral bulking, in accordance with an exemplary embodiment of the disclosure. 5A illustrates a side view of a device for periurethral bulking. 5B, 5C, and 5D each illustrate a detailed view of a locking mechanism of a device for periurethral bulking.

FIGS. 6A-6D illustrate various side views of a device for periurethral bulking, in accordance with an exemplary embodiment of the disclosure. 6A illustrates a side views of a device for periurethral bulking. 6B, 6C, and 6D each illustrate a detailed view of a ratcheting mechanism of a device for periurethral bulking.

FIGS. 7A, 7B, and 7C each illustrate various exploded perspective views of components related to a needle manifold of a device for periurethral bulking, in accordance with an exemplary embodiment of the disclosure.

### DEFINITIONS

The instant invention is most clearly understood with reference to the following definitions.

As used herein, the singular form “a,” “an,” and “the” include plural references unless the context clearly dictates otherwise.

As used in the specification and claims, the terms “comprises,” “comprising,” “containing,” “having,” and the like can have the meaning ascribed to them in U.S. patent law and can mean “includes,” “including,” and the like.

As used herein, the term “lateral” refers to a component that is located relatively away from the midline of a subject's body.

As used herein, the term “medial” refers to a component that is located between the proximal distal ends of a device.

The terms “proximal” and “distal” can refer to the position of a portion of a device relative to the remainder of the device or the opposing end as it appears in the drawing. The proximal end can be used to refer to the end manipulated by the user. The distal end can be used to refer to the end of the device that is inserted and advanced and is furthest away from the user. As will be appreciated by those skilled in the art, the use of proximal and distal could change in another context, e.g., the anatomical context in which proximal and distal use the patient as reference, or where the entry point is distal from the user.

Unless specifically stated or obvious from context, the term “or,” as used herein, is understood to be inclusive.

### DETAILED DESCRIPTION

It would be desirable to provide a device to treat SUI successfully without the use of a permanent mesh to address the foregoing problems and related problems in the art. More specifically, it would be desirable to provide a device for periurethral bulking.

The present disclosure relates to a device for periurethral bulking. In certain embodiments of the disclosure, a medical device (sometimes referred to herein as a “device for periurethral bulking” or “device”) is provided including a distal end, a proximal end, and a bridge attachment positioned between the distal end and the proximal end. In some embodiments, the distal end includes and/or is configured to receive or house a distal portion of a medical device (e.g., cystoscope) and at least one needle. In some embodiments, the proximal end includes and/or is configured to receive or house viewing optics of the medical device. In some embodiments, the bridge attachment is in fluid communication with the at least one needle.

In some embodiments, the distal end includes a sheath to house a cystoscope (or another medical device) with a optically transparent tip to allow viewing. The optically transparent tip may be detachable from or fixed to the sheath. Additionally or alternatively, the optically transparent tip may be tapered. The optically transparent tip contains outlet channels (also referred to as “through channels” or “needle channels”) to allow the deployment of the needle(s) for injecting a fluid into the urethral wall, periurethral space, or other tissue. For example, in some embodiments, the device includes one or more needles that run the length of the sheath and are deployed through the outlet channels. The optically transparent tip may contain any suitable number of outlet channels for any suitable number of needles. For example, in some embodiments, the optically transparent tip includes at least two (2) outlet channels corresponding to at least two (2) needles. In some embodiments, the optically transparent tip includes three (3) outlet channels corresponding to three (3) needles. The outlet channels may be configured around the edge of the sheath in equidistant proportions or have slightly offset spacing. In some embodiments, the tip contains a mirrored optical system in order to view the space behind the proximal end of the sheath, so that the needle injection sites are visible. The sheath can be connected to a cylindrical base, which houses the fluid injection system. In some embodiments, the cylindrical base has a handle with two grips.

A syringe may be fluidly coupled to the fluid injection system through any suitable fitting or attachment. For example, in some embodiments, the fluid injection system includes a port (e.g., an external Luer lock or other port) fluidly coupled to the base or bridge attachment for administering an external fluid from a syringe into the fluid injection system. In certain embodiments, a needle manifold may be included. The needle manifold may be a circular manifold housed within the injection system (e.g., within a handle of the cystoscope). In such embodiments, the needle manifold may be fluidly coupled to an external syringe through the fitting or attachment (e.g., a Luer lock connector) to facilitate filling of the manifold with any suitable material (e.g., a fluid, a bulking agent, etc.). Once filled, the needle manifold assists dispensing said material in equal volumes from the needles (e.g., three needles) that are attached to the manifold. For example, the fluid may follow a circular path around the needle manifold such that it is evenly dispersed into the injection needles.

In some embodiments, the needles are deployed through compression of the handle. The compression of the handle may deploy the needles through the channel and/or into surrounding tissue at any suitable angle. Suitable angles include, but are not limited to, between about 35 degrees and about 100 degrees, between about 45 degrees and about 90 degrees, about 90 degrees, or any suitable combination, sub-combination, range, or sub-range thereof. For example, in some embodiments, the needles are in a bent shape at the end so that they may enter tissue at approximately a 90-degree angle (or another angle). In certain embodiments, the needles may have spaced markers to measure the depth of tissue penetration when deployed. The needles can be curved and/or made of an alloy such as nitinol or another shape-memory metal (e.g., Nitinol, other shape-memory alloys, and the like). Additionally or alternatively, the needle tip can be beveled.

In certain embodiments of the present disclosure, the needles are housed inside the sheath until a safety mechanism arm (e.g., a locking mechanism) is disengaged by the user to allow the compression of the handle. In some embodiments, the locking mechanism (e.g., a safety mechanism) is integrated with a handle. For example, in some embodiments, the locking mechanism is situated between two grips attached to the cylindrical base of the device. In some embodiments, the locking mechanism includes and/or is coupled to a ratcheting mechanism employed to maintain the position of the needles (i.e., prevent further deployment or retraction) until released by the user. In such embodiments, the locking mechanism/ratcheting mechanism keeps the depth of injection steady while a liquid or a fluid passes through the needles (i.e., maintains the position of the needles during injection). For example, in some embodiments, the needles are deployed following release of the ratchet mechanism by the user. In some embodiments, the ratchet mechanism may include a graded ratchet system to prevent the needles from retracting once they have reached their desired length outside of the sheath. Additionally or alternatively, in some embodiments, a needle return spring is contained within the cylindrical base. In such embodiments, the needle return spring returns the deployed needles to their original position within the interior of the sheath when a safety arm is released, and ratchet cogs are disengaged.

In certain embodiments, the sheath tip is a optically transparent tapered cap configured to fit over an existing cystoscope that allows for clear viewing of tissues (e.g., the urethra, bladder, bladder neck, etc.). In certain embodiments, the sheath tip may be uniform without any channels. In certain embodiments, the sheath tip may contain channels to allow the passage of fluid or tools through the cap and into the urethra, or it may house an optical mirror system for viewing urethral tissue that is positioned below the cap that is not readily viewable otherwise. In certain embodiments, the sheath tip may be a detachable cap or incorporated as a continuous sheath. In certain embodiments, the sheath tip may be attached to a cystoscope base.

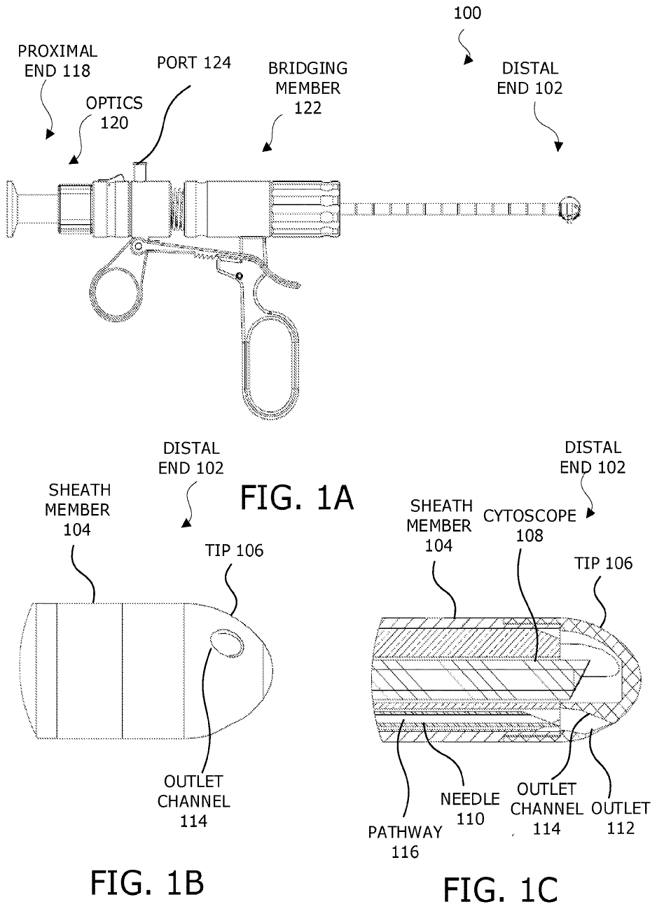

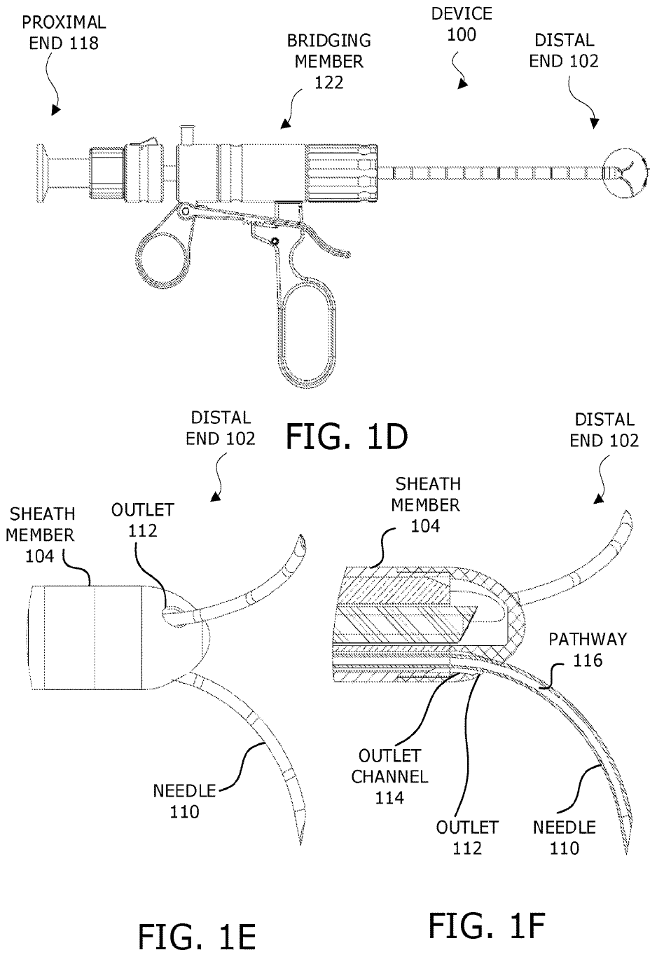

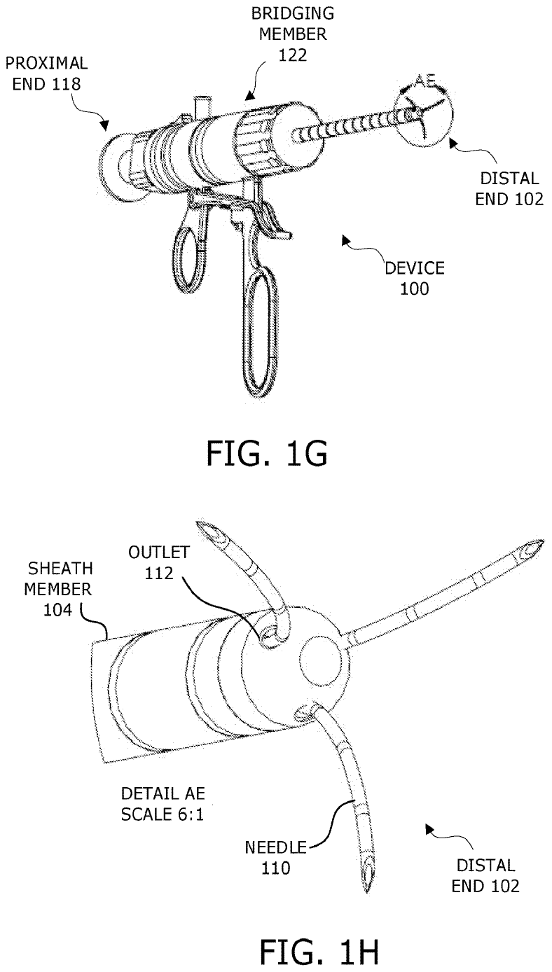

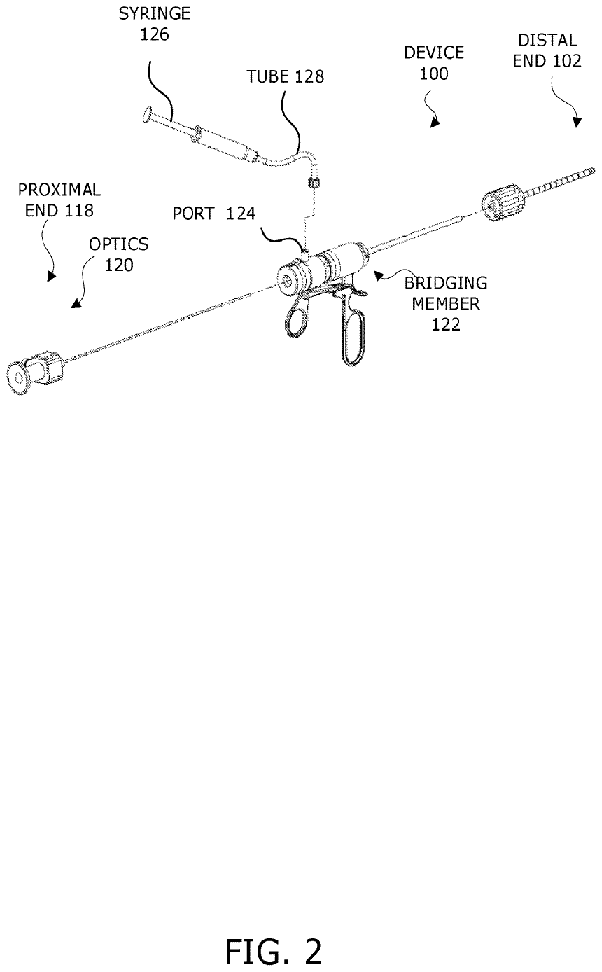

Referring now to FIGS. 1A-1C, a device 100 for periurethral bulking is illustrated. Device 100 is illustrated in an “undeployed” configuration (i.e., a configuration prior to or after needles are deployed). Device 100 includes a distal end 102. Distal end 102 includes a sheath member 104. Sheath member 104 includes a tip 106 (e.g., a optically transparent tip), located at a terminal end of sheath member 104. Tip 106 (e.g., optically transparent tip) tapers at distal end 102 of sheath member 104. In certain embodiments, tip 106 (e.g., optically transparent tip) is detachable from sheath member 104. In certain other embodiments, tip 106 (e.g., optically transparent tip) is not detachable from sheath member 104 (i.e., when tip 106 is permanently or semi-permanently attached to sheath 104). Sheath member 104 is configured to include a distal portion of cystoscope 108. Sheath member 104 is configured to include a plurality of needles 110. Each of the plurality of needles 110 defines a respective pathway 116 (e.g., a fluidic pathway). Pathway 116 may be used to transfer (e.g., flow) a substance (e.g., a fluid, a liquid, a bulking agent, water, saline solution, an analgesic, a gas, a bulking agent including platelet-rich plasma (PRP), a bulking agent including platelet-rich plasma (PRP) mixed with adipose tissue, etc.) to an end of needle 110. Each needle 110 (and its respective pathway 116) of the plurality of needles 110 are bendable to an angle such that each needle 110 (and its respective pathway 116) are configured to deploy into a tissue of a subject at an angle (e.g., of about 45-90 degrees relative to sheath member 104). Tip 106 (e.g., optically transparent tip) is configured to include a plurality of outlet channels 114. Each of the plurality of outlet channels 114 are configured such that each of the plurality of outlet channels 114 allows passage of only a single needle 110 from the plurality of needles 110. Each of the plurality of outlet channels 114 are configured to deploy the plurality of needles 110 (e.g., at a designated angle; at an angle of 30 degrees or greater relative to sheath member 104; at an angle of 45 degrees or greater relative to sheath member 104; at an angle of 90 degrees or greater relative to sheath member 104; at an angle of 105 degrees or greater relative to sheath member 104, etc.).

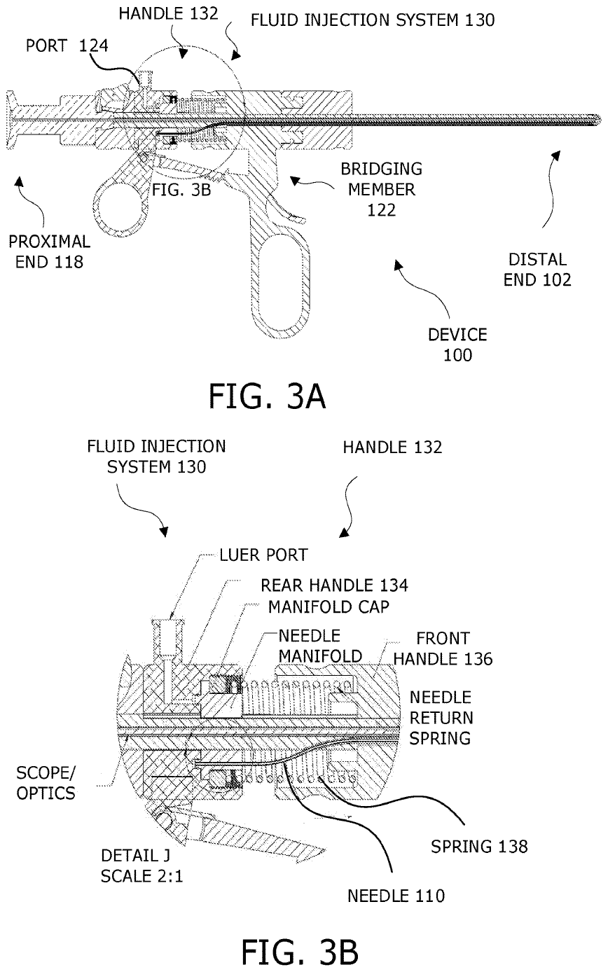

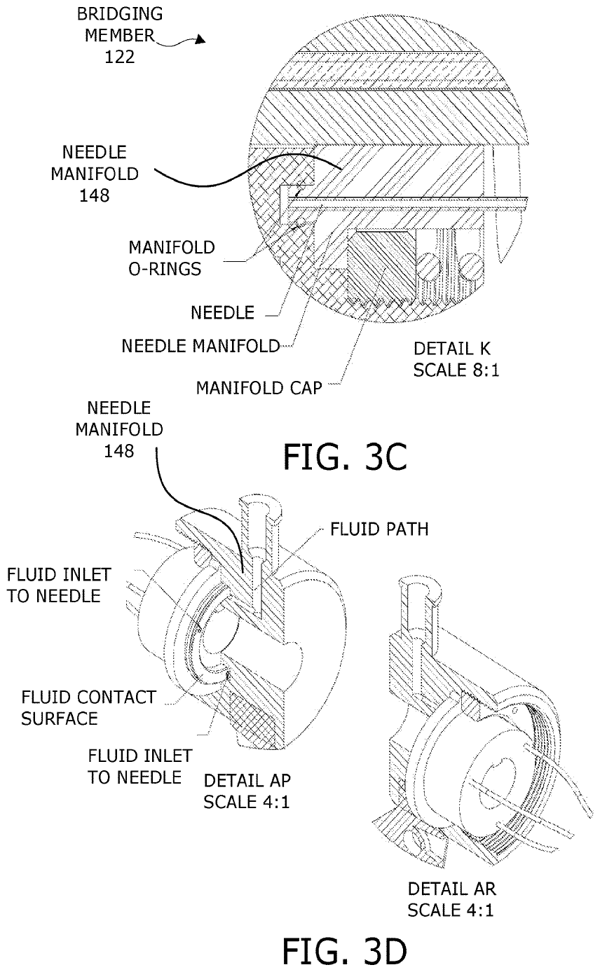

Device 100 also includes a proximal end 118, including optics 120 (e.g., viewing optics) of cystoscope 108. Device 100 also includes a bridge attachment 122 positioned in between distal end 102 and proximal end 118. The bridge attachment 122 is in fluidic communication with the plurality of needles 110. As illustrated in certain embodiments, a syringe (e.g., syringe 126 of FIG. 2) may be used to provide a material (e.g., a liquid, a bulking agent, water, saline solution, an analgesic, a gas, etc.) to needle 110 via bridge attachment 122 (e.g., via port 124). The bridge attachment 122 includes a fluid injection system 130 (described in connection with FIGS. 3A-3D).

Each needle 110 (and respective pathway 116) of the plurality of needles 110 are bendable to an angle such that each needle 110 (and respective pathway 116) is configured to deploy into a tissue of a subject (e.g., a patient) at an angle of about 45-90 degrees relative to sheath member 104. Each needle 110 may be made from a variety of materials (e.g., flexible super elastic nitinol, a shape retaining metal, a malleable metal alloy, etc.) to accomplish the desired deployment (e.g., bending at a desired angle when deployed). In some embodiments, the needles 110 can be configured (e.g., through geometry, shape memory, and the like) to deploy a distal injection orifice to a predefined depth (e.g., between about 1 cm and about 1.5 cm, about 1.5 cm, and the like) relative to an outer surface of the cystoscope.

Referring now to FIGS. 1D-1H, device 100 is illustrated in an “deployed” configuration (i.e., a configuration where needles are deployed). As illustrated in the detailed views of FIGS. 1E-IF, needle 110 and pathway 116 are deployed through outlet channel 114 of outlet 112. Needle 110 is illustrated at an angle of approximately 90 degrees relative to sheath member 104 (i.e., with respect to the longitudinal centerline of sheath member 104). As illustrated in the perspective views of FIGS. 1G-1H, device 100 includes three (3) needles 110. The plurality of needles 110 are arranged such that they circumscribe a distal portion of the cystoscope. In certain embodiments, the plurality of needles 110 may be arranged adjacent to the distal end of the cystoscope with unequal spacing between each of the plurality of needles. In certain embodiments, the plurality of needles 110 may be arranged in an asymmetrical pattern such that each needle in the plurality of needles is not necessarily equidistant from its neighboring needle In some exemplary uses of device 100, needle(s) 100 and associated outlet channel(s) 114 are configured to deploy into tissue to avoid selected blood vessels (e.g., major blood vessels).

For example, the angle of deployment, length of the deployed needle (e.g., overall needle length, free length, etc.), and/or geometry (e.g., needle point sharpness, gauge, bevel type, bevel length, lancet features, etc.) of the distal portion of the needle may be altered in accordance with the particular application (e.g., a procedure involving a female patient, a male patient, an adult patient, a child patient, a geriatric patient, a pregnant patient, etc.) of device 100. In certain embodiments and applications, the plurality of needles 110 may include beveled tips (e.g., A-bevel, B-bevel, C-bevel, Bias, Chiba, Crawford, Deflected Tip, Francine, Hustead, Huber, Trocar, Tuohey, etc.). The sharpness of the tips (and/or portions of tips, such as an edge of a beveled tip, a burr, etc.) of the plurality of needles 100 may be adjusted based on the anatomy of a patient or type of procedure (e.g., inpatient or outpatient procedure, local or general anesthesia, etc.).

In some embodiments, the radial arrangement of needles 110 can be defined with regard to a subject's anatomy. For example, the device 100 can be configured (e.g., with instructions, reference markings, and the like). The radial arrangement can be configured to avoid significant blood vessels around the urethra. In some embodiments, the needles 110 are arranged with 0° being the anterior (or “up” direction for a patient lying on her back) at: 0° (12:00), 60°(4: 00), and 120°(8:00).

Referring now to FIG. 2, an exploded view of device 100 and associated elements are illustrated. Specifically, syringe 126 is illustrated connected to a tube 128. Port 124 is configured to receive fluid from syringe 126 (e.g., a Luer lock type of syringe); thus, bridge attachment 122 is in fluidic communication with syringe 126. Since bridge attachment 122 is configured to be in fluidic communication with the plurality of needles 110, syringe 126 may be in fluidic communication (e.g., indirectly) with the plurality of needles 110.

Referring now to FIGS. 3A-3D, fluid injection system 130 of the bridge attachment 122 is illustrated. Fluid injection system 130 includes a port 124 (e.g., a Luer port) configured to receive a fluid (e.g., a liquid, a bulking agent, water, saline solution, an analgesic, a gas, a bulking agent including platelet-rich plasma (PRP), a bulking agent including platelet-rich plasma (PRP) mixed with adipose tissue, etc.) from a Luer lock type of syringe.

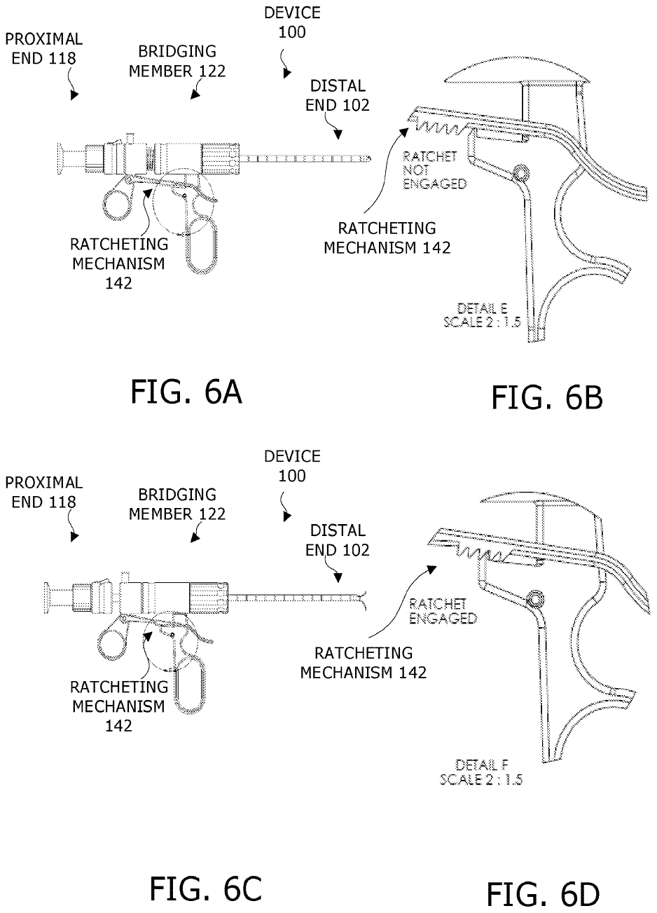

The bridge attachment 122 is illustrated to include a handle 132. Handle 132 may include multiple portions (e.g., multiple subcomponents, such as handles). In FIG. 3B, handle 132 is illustrated including a rear handle 134 (i.e., a handle portion near proximal end 118) and a front handle 136 (i.e., a handle portion near distal end 102). In certain embodiments, when handle 132 is compressed (e.g., when rear handle 134 is drawn towards front handle 136, or vice versa) a spring 138 (e.g., a return spring) is compressed. As illustrated in FIG. 3B, needle 110 may be coupled to rear handle 134 such that, when handle 132 is compressed, at least one of the plurality of needles 110 is deployed (e.g., into tissue). Once at least one of the plurality of needles 110 is deployed, the needle(s) may be locked in place by a ratchet mechanism to prevent further deployment or retraction (e.g., see ratchet mechanism 142 of FIGS. 6A-6D). When handle 132 is decompressed (e.g., when the spring force of spring 138 restores handle 132 to an uncompressed state), at least one of the plurality of needles 110) retracts into sheath member 104 of device 100.

Referring now to FIGS. 3C-3D, a needle manifold 148 of the bridge attachment 122 is included. Needle manifold 148 is a circular manifold housed within fluid injection system 130 within handle 132 (e.g., rear handle 134). Needle manifold 148 is a hollow circular cavity that is filled with a material (e.g., a fluid, a bulking agent, etc.) by an external syringe attached by a Luer lock connector (e.g., a fitting, an attachment, etc.). Once filled, needle manifold 148 assists dispensing said material (e.g., a fluid) in equal volumes from needles (e.g., three needles) that are attached to the manifold.

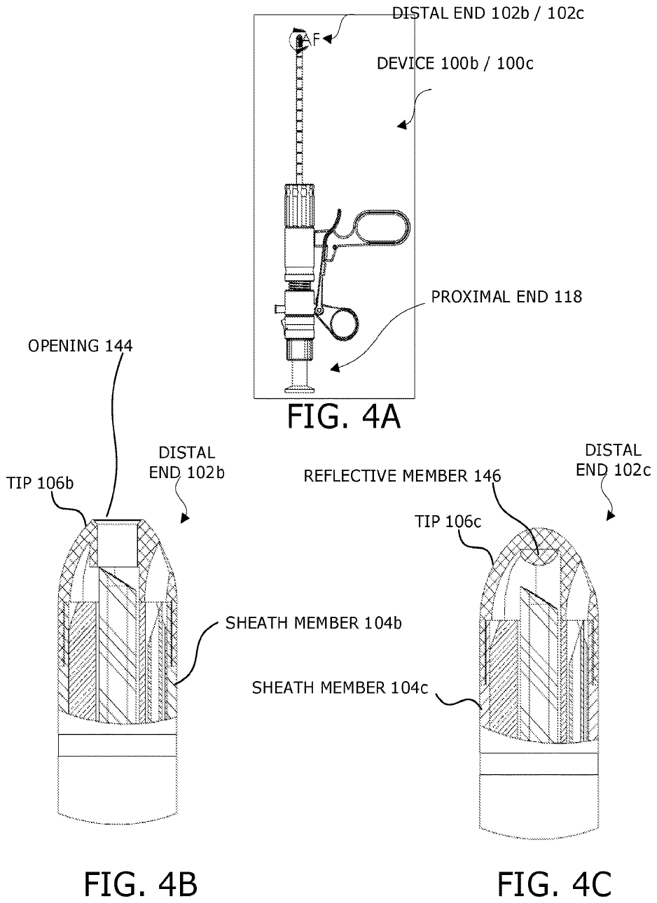

Referring now to FIGS. 4A-4C, various embodiments of tips of devices 400b and 400c are illustrated. Referring specifically to FIG. 4A, device 100b and device 100c are illustrated. Device 100b and 100c are substantially the same as device 100 (unless clearly or explicitly indicated otherwise) except that they respectively include distal ends 102b and 102c (including tips 106b and 106c, respectively) in lieu of distal end 102 (including tip 106). Tips 106b and 106c (e.g., optically transparent tips) are similar in that they both taper at distal ends 102b and 102c of the sheath member 104b and 104c, respectively.

Referring now to FIG. 4B, distal end 102b include tip 106b is illustrated. Distal end 106b is substantially similar to distal end 106 (e.g., of FIGS. 1B-1C) except an opening 144 is included (e.g., for a cystoscope to pass through during a procedure, for the injection of a material, etc.). Referring now to FIG. 4C, tip 106c (e.g., optically transparent tip) includes (optionally) a reflective member 146 (e.g., a curved, convex, or concave position for viewing the urethral tissue at a proximal end 118 of the sheath member 104c.

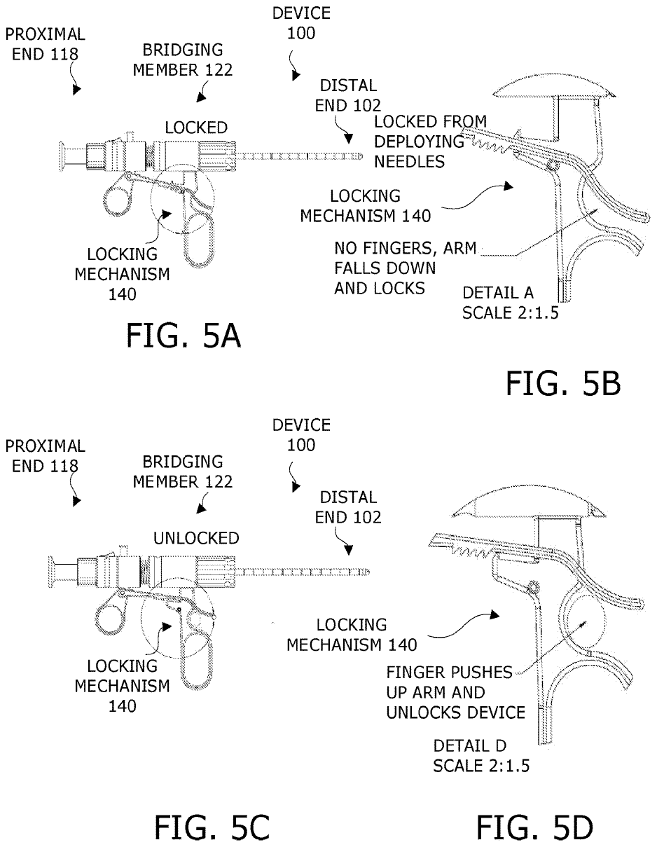

Referring now to FIGS. 5A-5D, locking mechanism 140 is illustrated. Referring specifically to FIGS. 5A-5B, device 100 is illustrated in a “locked” configuration. Referring specifically to FIGS. 5C-5D, device 100 is illustrated in an “unlocked” configuration. As illustrated, a finger of a user may be used to press a portion of locking mechanism 140 towards the bridge attchment 122 such that locking mechanism unlocks.

Referring now to FIGS. 6A-6D, ratcheting mechanism 142 is illustrated. Referring specifically to FIGS. 6A-6B, device 100 is illustrated in a configuration where ratcheting mechanism 142 is not engaged. Referring specifically to FIGS. 6C-6D, device 100 is illustrated in a configuration where ratcheting mechanism 142 is not engaged.

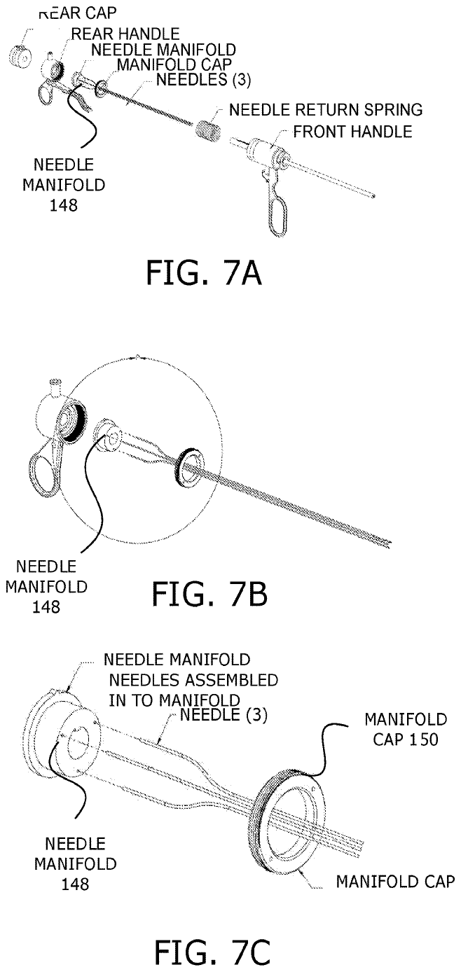

Referring now to FIGS. 7A-7C, needle manifold 148 and associated components are illustrated. Needle manifold 148 is a circular manifold housed within an injection system (e.g., a fluid injection system) within handle (e.g., rear handle 134). Needle manifold 148 is a hollow circular cavity that is filled with a material (e.g., a fluid, a bulking agent, etc.) by an external syringe attached by a Luer lock connector (e.g., a fitting, an attachment, etc.). Needle manifold 148 is mated with manifold cap 150 to contain fluids. Once filled, needle manifold 148 assists dispensing said material (e.g., a fluid) in equal volumes from needles (e.g., three needles) that are attached to the manifold.

Although several exemplary embodiments illustrate a device with three (3) needles (e.g., FIGS. 1G-1H), the breadth of the disclosure is not so limited. In certain exemplary embodiments, device 100 may include one (1) needle, two (2) needles, three (3) needles, four (4) needles, five (5) needles, six (6) needles, and so on. In other exemplary embodiments, device may include more needles than are deployed; in other words, the needles may be independently controllable from one another and/or the total number of needles deployed may be less than the total number of needles contained in the plurality of needles.

In connection with the various exemplary embodiments of the drawings, a method of using certain of the embodiments is disclosed. The method for viewing and simultaneously injecting a bulking material at multiple locations in the urethra of a subject, comprises injecting a bulking agent into the urethra of the subject using a device described herein (i.e., one of the embodiments of the present disclosure).

### Equivalents

Although preferred embodiments of the invention have been described using specific terms, such description is for illustrative purposes only, and it is to be understood that changes and variations may be made without departing from the spirit or scope of the following claims.

### Enumerated Embodiments

The following exemplary embodiments are provided, the numbering of which is not to be construed as designating levels of importance:

Embodiment 1 provides a device for periurethral bulking comprising:
a distal end comprising a sheath member having a optically transparent tip, wherein the sheath member is configured to include:
   a) a distal portion of a cystoscope; and
   b) a plurality of needles, wherein each of the plurality of needles includes a respective pathway;
a proximal end comprising viewing optics of the cystoscope; and
a bridge attachment positioned in between the distal end and the proximal end, wherein the bridge attachment is in fluidic communication with the plurality of needles;
wherein the optically transparent tip comprises a plurality of outlet channels such that each of the plurality of outlet channels allows passage of only a single needle from the plurality of needles;
wherein each of the plurality of outlet channels is configured to guide a respective needle to an angle of about 45-90 degrees relative to the sheath member;
wherein each needle and the respective pathway of the plurality of needles are bendable to an angle such that each needle and the respective pathway are configured to deploy into a tissue of a subject at an angle of about 45-90 degrees relative to the sheath member.

Embodiment 2 provides the device of embodiment 1, wherein the optically transparent tip tapers at the distal end of the sheath member.

Embodiment 3 provides the device of embodiments 1-2, wherein the optically transparent tip comprises a reflective member positioned for viewing urethral tissue at a proximal end of the sheath member.

Embodiment 4 provides the device of embodiments 1-3, wherein the optically transparent tip is detachable from the sheath member.

Embodiment 5 provides the device of embodiments 1-4, wherein the optically transparent tip is not detachable from the sheath member.

Embodiment 6 provides the device of embodiments 1-5, wherein the bridge attachment comprises a fluid injection system.

Embodiment 7 provides the device of embodiments 1-6, wherein the fluid injection system comprises a port configured to receive a fluid(s).

Embodiment 8 provides the device of embodiments 1-7, wherein the port is configured to receive the fluid from a Luer lock type of syringe.

Embodiment 9 provides the device of embodiments 1-8, wherein the fluid is one selected from the group consisting of a bulking agent, water, saline solution, and an analgesic.

Embodiment 10 provides the device of embodiments 1-9, wherein the bulking agent includes platelet-rich plasma (PRP) alone or mixed with adipose tissue.

Embodiment 11 provides the device of embodiments 1-10, wherein the bridge attachment further comprises a handle.

Embodiment 12 provides the device of embodiments 1-11, wherein when the handle is compressed at least one of the plurality of needles is deployed into the tissue.

Embodiment 13 provides the device of embodiments 1-12, wherein once deployed the plurality of needles are locked in place by a ratchet mechanism to prevent further deployment or retraction.

Embodiment 14 provides the device of embodiments 1-13, wherein when the handle is decompressed, the plurality of needles retracts into the sheath member.

Embodiment 15 provides the device of embodiments 1-14, wherein the bridge attachment further comprises a needle manifold that facilitates even distribution of the fluid to each needle in the plurality of needles.

Embodiment 16 provides the device of embodiments 1-15, wherein the plurality of needles are arranged to circumscribe the distal portion of the cystoscope.

Embodiment 17 provides the device of embodiments 1-16, wherein the plurality of needles are arranged adjacent to the distal end of the cystoscope with unequal spacing between each of the plurality of needles.

Embodiment 18 provides the device of embodiments 1-17, wherein the plurality of needles are arranged in an asymmetrical pattern such that each needle in the plurality of needles is not necessarily equidistant from its neighboring needle.

Embodiment 19 provides the device of embodiments 1-18, wherein the plurality of needles are configured to deploy into the tissue such that the plurality of needles avoid selected blood vessels.

Embodiment 20 provides the device of embodiments 1-19, wherein the plurality of needles comprises from about 2 to about 5 needles.

Embodiment 21 provides the device of embodiments 1-20, wherein the plurality of needles comprises 3 needles.

Embodiment 22 provides the device of embodiments 1-21, wherein the plurality of needles comprises beveled tips.

Embodiment 23 provides a method for viewing and simultaneously injecting a bulking material at multiple locations in the urethra of a subject, the method comprising injecting a bulking agent into a urethra of the subject using the device of embodiments 1-22.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A device for periurethral bulking comprising:
a distal end comprising a sheath member having a optically transparent tip, wherein the sheath member is configured to include:
a) a distal portion of a cystoscope; and
b) a plurality of needles, wherein each of the plurality of needles includes a respective pathway;
a proximal end comprising viewing optics of the cystoscope; and
a bridge attachment positioned in between the distal end and the proximal end, wherein the bridge attachment is in fluidic communication with the plurality of needles;
wherein the optically transparent tip comprises a plurality of outlet channels such that each of the plurality of outlet channels allows passage of only a single needle from the plurality of needles;
wherein each of the plurality of outlet channels is configured to guide a respective needle to an angle of about 45-90 degrees relative to the sheath member;
wherein each needle and the respective pathway of the plurality of needles are bendable to an angle such that each needle and the respective pathway are configured to deploy into a tissue of a subject at an angle of about 45-90 degrees relative to the sheath member.

2. The device of claim 1, wherein the optically transparent tip tapers at the distal end of the sheath member.

3. The device of claim 2, wherein the optically transparent tip comprises a reflective member positioned for viewing urethral tissue at a proximal end of the sheath member.

4. The device of claim 1, wherein the optically transparent tip is detachable from the sheath member.

5. The device of claim 1, wherein the optically transparent tip is not detachable from the sheath member.

6. The device of claim 1, wherein the bridge attachment comprises a fluid injection system.

7. The device of claim 6, wherein the fluid injection system comprises a port configured to receive a fluid.

8. The device of claim 7, wherein the port is configured to receive the fluid from a Luer lock type of syringe.

9. The device of claim 1, wherein the fluid is one selected from the group consisting of a bulking agent, water, saline solution, and an analgesic.

10. The device of claim 9, wherein the bulking agent includes platelet-rich plasma (PRP) alone or mixed with adipose tissue.

11. The device of claim 1, wherein the bridge attachment further comprises a handle.

12. The device of claim 11, wherein when the handle is compressed at least one of the plurality of needles is deployed into the tissue.

13. The device of claim 12, wherein once deployed the plurality of needles are locked in place by a ratchet mechanism to prevent further deployment or retraction.

14. The device of claim 12, wherein when the handle is decompressed, the plurality of needles retracts into the sheath member.

15. The device of claim 1, wherein the bridge attachment further comprises a needle manifold that facilitates even distribution of the fluid to each needle in the plurality of needles.

16. The device of claim 1, wherein the plurality of needles are arranged to circumscribe the distal portion of the cystoscope.

17. The device of claim 1, wherein the plurality of needles are arranged adjacent to the distal end of the cystoscope with unequal spacing between each of the plurality of needles.

18. The device of claim 1, wherein the plurality of needles are arranged in an asymmetrical pattern such that each needle in the plurality of needles is not necessarily equidistant from its neighboring needle.

19. The device of claim 18, wherein the plurality of needles are configured to deploy into the tissue such that the plurality of needles avoid selected blood vessels.

20. The device of claim 1, wherein the plurality of needles comprises from about 2 to about 5 needles.

21. The device of claim 1, wherein the plurality of needles comprises 3 needles.

22. The device of claim 1, wherein the plurality of needles comprises beveled tips.

23. A method for viewing and simultaneously injecting a bulking material at multiple locations in the urethra of a subject, the method comprising injecting a bulking agent into a urethra of the subject using the device of claim 1.
